(19) United States
(12) Patent Application Publication
(10) Pub. No.: US 2021/0280280 A1
(43) Pub. Date: Sep. 9, 2021

(54) Systems And Methods For Dynamically Providing On-Demand Behavioral Insights

(71) Applicants: Raghavendra Misra, Ponte Vedra, FL (US); Rohan Misra, Ponte Vedra, FL (US)

(72) Inventors: Raghavendra Misra, Ponte Vedra, FL (US); Rohan Misra, Ponte Vedra, FL (US)

(21) Appl. No.: 17/194,211

(22) Filed: Mar. 6, 2021

Related U.S. Application Data
(60) Provisional application No. 62/986,537, filed on Mar. 6, 2020.

(51) Int. Cl. G16H 10/20

(52) U.S. Cl. CPC G16H 10/20

(57) ABSTRACT

The present disclosure provides generally for providing behavioral insights across individuals and groups and presenting this information on demand. In some embodiments, the behavioral insight device may create and distribute relevant information across a system to provide quick and accurate ODBI's for various scenarios. In some implementations, the device may provide an accurate psychometric profile data of each profile. In some aspects, this device may relate to systems and methods for providing on-demand dynamic behavioral insights for individuals and groups regarding different events and scenarios requested in an ODBI. In some embodiments, the BIVA system may automatically update and use outside resources to implement different information across the system.

### CROSS REFERENCE TO RELATED APPLICATION

This application claims priority to and the full benefit of U.S. Provisional Patent Application Ser. No. 62/986,537, filed Mar. 6, 2020, and titled “SYSTEMS AND METHODS FOR DYNAMICALLY PROVIDING ON-DEMAND BEHAVIORAL INSIGHTS”, the entire contents of which are incorporated in this application by reference.

### BACKGROUND OF THE DISCLOSURE

Psychometrics were developed as a method for measuring psychological attributes or abilities, typically through an assessment. The study of psychometrics includes the study of the statistical, mathematical, and professional protocols that underpin any assessment used and how any assessment is constructed, used, and evaluated.

Some of the very first instances of mental testing started in ancient China, where proficiency assessments were used to grade, rank, and place personnel. These early assessments were a mix of skill, intelligence, and endurance testing, often requiring a candidate to attend testing for a full day and night. At the time, these exams had incredibly low passage rates, with a stated goal of only finding the best personnel for public official positions.

In the 13^{th} century, European universities gave students formal oral testing, with written examinations starting in the 16^{th} century. By the beginning of the 19^{th} century, competitive university examinations were instituted. As social mobility increased, society relied more on other forms of assessments to determine who was appropriate for certain roles, such as in the government. This switch from personal judgment or word of mouth to something more impartial helped streamline application processes and vetting for whether a person was fit for a position or role in a group. This led to an academic interest in human variation and measurement-based psychology.

The interest in measuring behavioral capacity and mental capabilities has expanded to determining who might be appropriate when building a team within an organization. A version of a psychometric assessment might be useful when determining a culture fit or how to best optimize the makeup of a team. For example, these assessments may vary and test for aptitude, ability, or personality. Employers may use these at any phase of an employee's life cycle within a company, from onboarding, determining fit, and future potential, such as for a promotion or working in a different department or specialty.

One particular focus of psychometrics is providing objective and impersonal assessments for individuals or groups to be compared without unconscious bias. Despite the advancements made standardizing and optimizing assessments within an organization, there is always room for increased efficiency when implementing or reviewing psychometric assessments. Further, personality insights are provided through complex graphs and dashboards, requiring extensive review of a profile to extract meaningful information.

As technology relating to psychometric analysis continues to evolve, more information and variables are being integrated on a broader level to provide more accurate and on-point information. For example, how one does psychometric analysis has shifted from being done in person or on paper to being done digitally or through a computer. Psychometric analysis could be richly improved if it were able to pull even more data from various areas or harness currently existing technology to enrich the feedback or insight the process may provide. However, there is still difficulty in receiving on-demand psychometric analysis via oral or verbal requests or prompts.

### SUMMARY OF THE DISCLOSURE

What is needed is a method and system for providing on-demand behavioral insights for individuals and groups. The method and system may efficiently and effectively provide summary psychometric profile data. Accordingly, the present disclosure relates to systems and methods for providing on-demand dynamic behavioral insights for individuals and groups. For example, tapping into an existing infrastructure involving intelligent virtual assistants or intelligent personal assistants that could receive and interpret human speech or other inputs.

In some embodiments, a system may provide a virtual assistant that could passively receive information relating to an individual or a group of individuals. In some implementations, the virtual assistant may passively analyze any received input to run psychometric analysis on the received information. In some aspects, the virtual assistant may use variables relating to someone's voice, such as tone, pitch, or volume, to integrate into a psychometric analysis.

In some embodiments, the virtual assistant may respond to verbal or oral prompts requesting psychometric information about an individual or a group. In some implementations, the virtual assistant may provide on-demand analysis about anyone. In some aspects, the system may provide regular updates based on information received about potential team dynamics. In some embodiments, the system may provide recommendations based on passive information received to improve group dynamics or interactions between specific individuals. In some implementations, the system may create profiles based on who interacts with the virtual assistant.

In some embodiments, a system of one or more computers may be configured to perform particular operations or actions by virtue of having software, firmware, hardware, or a combination of them installed on the system that in operation causes or cause the system to perform the actions. In some implementations, one or more computer programs may be configured to perform particular operations or actions by virtue of including instructions that, when executed by data processing apparatus, cause the apparatus to perform the actions. In some aspects, one general aspect may include a computer-implemented method for providing on-demand behavioral insights.

In some embodiments, the computer-implemented method may further may comprise receiving an activation prompt requesting logical access to behavioral insights of a subject, where the activation prompt may be received through a behavioral insight virtual assistant; receiving a communication destination; receiving an identifier of the subject; receiving a purpose for accessing behavioral insights of the subject; accessing a subject profile through at least one database, where the subject profile may comprise at least the identifier and behavioral insights of the subject; generating on-demand behavioral insights, where the on-demand behavioral insights may be based on predefined threshold parameters comparing the purpose to the subject profile; and communicating on-demand behavioral insights to the communication destination. In some implementations, the computer-implemented method may include corresponding computer systems, apparatus, and computer programs recorded on one or more computer storage devices, each configured to perform the actions of the methods.

In some aspects, behavioral insights of the subject may comprise a plurality of behavioral insight types, where the predefined threshold parameters may comprise at least one requested behavioral insight type from the plurality of behavioral insight types. In some embodiments, the subject may comprise a plurality of individuals. In some implementations, behavioral insights of the subject may comprise an aggregation of behavioral insights of the plurality of individuals. In some aspects, behavioral insights of the subject may comprise a summary of behavioral insights of the plurality of individuals. In some embodiments, on-demand behavioral insights may be provided based on predefined format requirements. In some implementations, predefined format requirements may be based at least in part on behavioral insights associated with the requestor. In some aspects, predefined threshold parameters may be based on the weighted value, and where the format is based at least in part on the weighted value. In some embodiments, the described techniques may include hardware, a method or process, or computer software on a computer-accessible medium.

In some implementations, the system may include a system for providing on-demand behavioral insights. In some aspects. the system may further comprise a behavioral insight virtual assistant, a behavioral insight server, two or more of: a psychometric profile database, a skills database, a personal information database. In some embodiments, the system may further comprise an executable software, where the executable software instructs the behavioral insights server to: receive an activation prompt requesting logical access to behavioral insights of a subject, where the activation prompt is received through a behavioral insight virtual assistant; receive a communication destination; receive an identifier of the subject; receive a purpose for accessing behavioral insights of the subject; receive a requestor profile through at least one database access a subject profile through at least one database, where the subject profile may comprise at least the identifier and behavioral insights of the subject; generate on-demand behavioral insights, where the on-demand behavioral insights may be based on predefined threshold parameters comparing the purpose to the subject profile; and communicate on-demand behavioral insights to the communication destination. In some implementations, computer systems, apparatus, and computer programs recorded on one or more computer storage devices, may be configured to perform the actions of the methods.

In some aspects, behavioral insights may be provided based on predefined format requirements. In some embodiments, the executable software may instruct the behavioral insight server to receive a requestor profile may comprise behavioral insights of a requestor of the activation prompt, where predefined format requirements may be based at least in part on behavioral insights associated with the requestor. In some implementations, the executable software instructs the behavioral insight server to assign a weighted value to behavioral insights of the subject, where predefined threshold parameters may be based on the weighted value, and where the format is based at least in part on the weighted value. In some aspects, the executable software may be configured to logically interface with a behavioral insight virtual assistant. In some embodiments, the behavioral insight virtual assistant may comprise the communication destination. In some implementations, the on-demand behavioral insights may be delivered as audio. In some aspects, external hardware may be accessible to the behavioral insight virtual assistant. In some embodiments, on-demand behavioral insights may direct behavioral insight virtual assistant external hardware interaction. In some implementations, requesting logical access to behavioral insights may be received as audio.

### BRIEF DESCRIPTION OF THE DRAWINGS

The accompanying drawings, that are incorporated in and constitute a part of this specification, illustrate several embodiments of the disclosure and, together with the description, serve to explain the principles of the disclosure:

FIG. 1A illustrates exemplary process steps for requesting and receiving ODBI from BIVA, according to some embodiments of the present disclosure.

FIG. 1B illustrates exemplary access flow for requesting and receiving ODBI from BIVA, according to some embodiments of the present disclosure.

FIG. 2A illustrates exemplary process steps for receiving and providing ODBI, according to some embodiments of the present disclosure.

FIG. 2B illustrates exemplary process steps for developing and providing ODBI, according to some embodiments of the present disclosure.

FIG. 2C illustrates exemplary process steps for collecting data that may be used for ODBI, according to some embodiments of the present disclosure.

FIG. 3A illustrates exemplary sources of information that BIVA may access to develop and provide ODBI, according to some embodiments of the present disclosure.

FIG. 3B illustrates exemplary extraction steps for identifying relevant data to develop ODBI, according to some embodiments of the present disclosure.

FIG. 4A illustrates exemplary process steps for processing a request for ODBI, wherein the ODBI may be customized based on behavioral insights of the requestor.

FIG. 4B illustrates exemplary process steps for customizing ODBI based on behavioral insights of the requestor, according to some embodiments of the present disclosure.

FIG. 5 illustrates an exemplary behavioral insight dashboard for an individual, according to some embodiments of the present disclosure.

FIG. 6A illustrates an exemplary team behavioral insight dashboard, according to some embodiments of the present disclosure.

FIG. 6B illustrates exemplary behavioral insights for a team, wherein the behavioral insights are based at least in part on psychometric profiles.

FIG. 7 illustrates exemplary behavioral insights and meeting strategy suggestions, according to some embodiments of the present disclosure.

FIG. 8 illustrates exemplary behavioral insights within a meeting, according to some embodiments of the present disclosure.

FIG. 9 illustrates exemplary behavioral insights within a meeting, according to some embodiments of the present disclosure.

FIG. 10 illustrates exemplary behavioral insights within a discussion, according to some embodiments of the present disclosure.

FIG. 11A illustrates an exemplary ODBI for a user, according to some embodiments of the present disclosure.

FIG. 11B illustrates an exemplary ODBI for a user, according to some embodiments of the present disclosure.

FIG. 12 illustrates exemplary distribution of behavioral insights within an organization, according to some embodiments of the present disclosure.

FIG. 13 illustrates an exemplary processing and interface system, according to some embodiments of the present disclosure.

FIG. 14 illustrates an exemplary block diagram of an exemplary embodiment of a mobile device.

FIG. 15 illustrates a plurality of exemplary BIVA, according to some embodiments of the present disclosure.

FIG. 16 illustrates an exemplary BIVA with a plurality of notification types, according to some embodiments of the present disclosure.

FIG. 17 illustrates an exemplary flowchart for collecting and delivering ODBI, according to some embodiments of the present disclosure.

FIG. 18 illustrates an exemplary flowchart for collecting and delivering ODBI, according to some embodiments of the present disclosure.

FIG. 19 illustrates exemplary method steps for receiving an ODBI request and providing an ODBI, according to some embodiments of the present disclosure.

FIG. 20 illustrates exemplary methods steps for receiving an ODBI request and providing an ODBI, according to some embodiments of the present disclosure.

### DETAILED DESCRIPTION

The present disclosure provides generally for providing behavioral insights across individuals and groups and presenting this information in a clear, actionable, and reliable way. The method and system may efficiently and effectively assess individuals and groups using a variety of factors, such as with psychometric assessments. Accordingly, the present disclosure relates to systems and methods for providing dynamic behavioral insights for individuals and groups.

In the following sections, detailed descriptions of examples and methods of the disclosure will be given. The description of both preferred and alternative examples though thorough are exemplary only, and it is understood that to those skilled in the art variations, modifications, and alterations may be apparent. It is therefore to be understood that the examples do not limit the broadness of the aspects of the underlying disclosure as defined by the claims.

Glossary
On-Demand Behavioral Insight (ODBI): as used herein refers to characteristics and attributes associated with a person, persons, or groups that may be conveyed and provided upon request. In some aspects, the request may occur orally, such as through a virtual assistant AI or cloud-based voice service. In some embodiments, the characteristics and attributes may be a combination of psychometric profile, interests, aspirations, talents, and skills, as non-limiting examples. In some aspects, ODBI may consider performance data, feedback data, historical data, user-generated data, or combinations thereof. In some aspects, ODBI may be provided based on the specific use case associated with the request, such as in preparation for an interview with a person or in preparation for a team meeting.
Behavioral Insight Virtual Assistant (BIVA): as used herein refers to the provider of on-demand behavioral insight (ODBI). In some embodiments, a BIVA may integrate with pre-existing virtual assistant AI services, such as Amazon's Alexa, Apple's Siri, Microsoft's Cortana, or Google's Google Assistant. In some implementations, a BIVA may be a standalone service that may be accessed through a communications network.
Psychometric Profile: as used herein refers to a set of psychometric attributes associated with a person, persons, or groups based on actual psychometric assessments, historical psychometric assessments associated with a demographic, implied psychometric attributes based on behavior, skills, interests, talents, aspirations and other information as gathered from recent social media posts, or combinations thereof.
Subject: as used herein refers to a person or group who is the subject of a psychometric profile, behavioral insights, feedback, or performance data, as non-limiting examples.

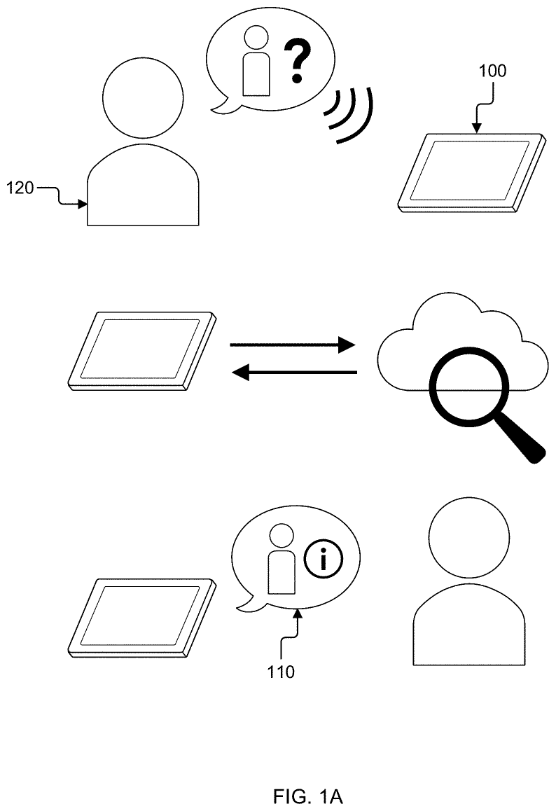

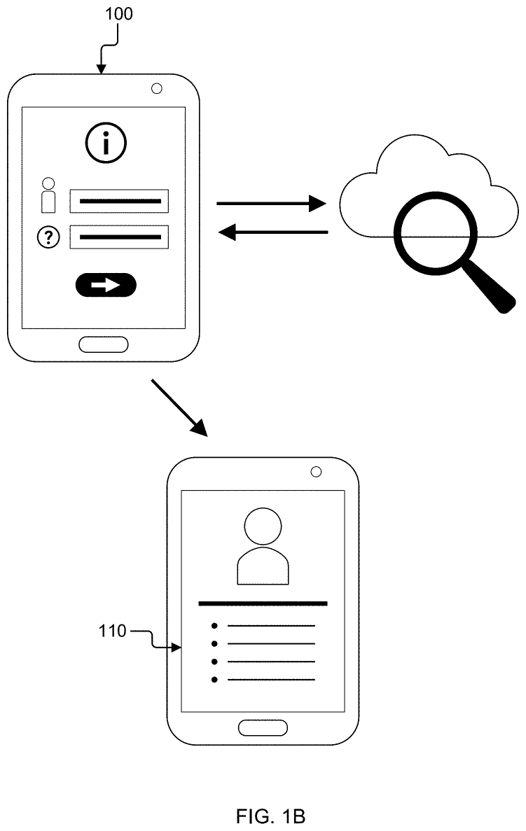

Referring now to FIG. 1A, exemplary process steps for requesting and receiving ODBI from BIVA are illustrated. In some implementations, a BIVA 100 may search for relevant profiles when a user 120 requests specific ODBI 110. In some aspects, a user 120 may request ODBI about a specific profile and a BIVA 100 may pull relevant information directly from a specific profile located in a storage system within the device.

In some embodiments, the storage device may be a cloud system storage where profiles and relevant ODBI 110 data may be uploaded to an online storage system within a network. For example, once a profile has been uploaded and saved onto the device, it may then be copied onto an online cloud storage system connected through the internet. This may prevent personal information from being lost, misplaced or accidentally deleted. In some aspects, a built-in storage system may be implemented, which may comprise a capped storage space. For example, a storage device may be equipped with a standard 64 gigabyte storage system, which may retain the most recent ODBI for easy reference. This may allow a requestor to return to the ODBI later without requiring an internet connection.

In some aspects, a BIVA 100 may have voice recognition and may identify the requestor, which may allow for more precise ODBI 110 without requiring extensive ODBI requests. For example, if the user 120 were to say, “Tell me more about Mary,” then the BIVA 100 may recognize who the requestor is and who a relevant Mary may be to that requestor. In some aspects, a BIVA 100 may be prompt the user 120 to select information manually by using the device. For example, the user 120 may manually select what profile and relevant information the BIVA 100 may provide.

In some embodiments, a BIVA may be programmed to only search for relevant information based on the ODBI requested. For example, a profile may be searched based on the ODBI the user 120 requests from the BIVA. In some implementations, once the BIVA 100 has pulled the relevant information it may transmit the ODBI 110 to the user 120. In some aspects, BIVA 100 may extract ODBI for a subject based on tangential profiles, such as the subject's friends or colleagues.

In some embodiments, the BIVA 100 may provide the ODBI 110 visually, orally, or both. In some implementations, the BIVA 100 may provide the ODBI 110 visually to the user 120 via the screen of the device. In some aspects, the information may be shown as a list or in different forms for the user 120 to best understand, or the BIVA 100 may show the entire profile of the requested ODBI 110. In some aspects, the BIVA 100 may orally tell the user 120 all of the relevant information in a no specific order. In some embodiments, BIVA 100 may orally provide ODBI in a summary or shortened form and visually provide more detailed information, such as presenting the actual profile, for the requestor's reference.

Referring now to FIG. 1B, exemplary access flow for requesting and receiving ODBI from BIVA is illustrated. In some embodiments, a BIVA 100 may be accessed through a portable device 130, which may access cloud storage to provide ODBI 110 to the user 120. In some aspects, a BIVA 100 may access cloud storage or information to retrieve relevant information. In some embodiments, the portable device 130 may be directly connected to the BIVA 100 through an internet connection or Bluetooth.

In some implementations, the BIVA 100 may transmit the ODBI visually, orally or both as previously stated. In some embodiments, a BIVA 100 may only provide the ODBI 110 visually to the user 120 via the screen of the device. In some aspects, the information may be shown as a list or in different forms for the user 120 to best understand, or the BIVA 100 may show the entire profile of the requested ODBI 110. In some aspects, the BIVA 100 may orally tell the user 120 all of the relevant information in a no specific order. In some implementations, BIVA 100 may offer different levels of ODBI 110, such as brief, summary, or extensive, which may allow a requestor to determine the level of detail and length of the ODBI 110.

In some embodiments, the portable device 130 may access an individual profile for the ODBI 110. In some implementations, ODBI 110 may be requested for a group. BIVA 100 may access individual profiles and provide group ODBI based on collected and summarized individual data. In some aspects, BIVA 100 may access group profiles that may be developed separately. In some aspects, the ODBI 110 may identify each individual profile to create a group profile to provide the user 120 with a complete ODBI 110.

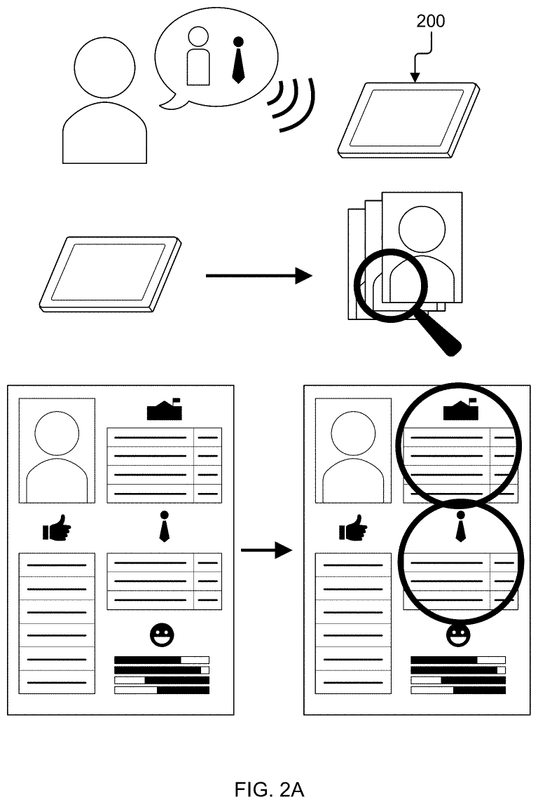

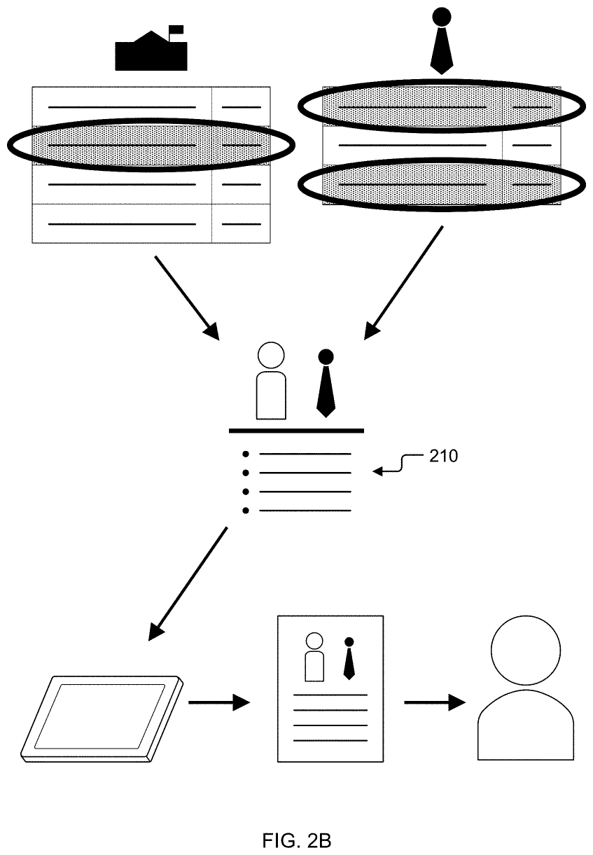

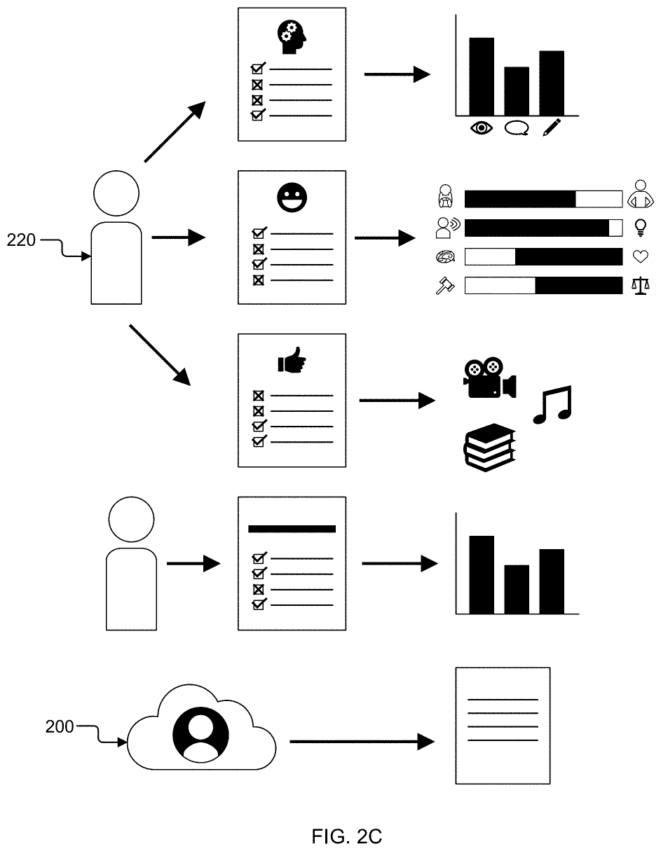

Referring now to FIG. 2A, exemplary process steps for receiving and providing ODBI are illustrated. In some implementations, a BIVA 200 may be accessed and may receive a ODBI request. In some embodiments, a BIVA 200 may access subject profiles and information to help retain more relevant information for the ODBI 210. In some aspects, the BIVA 200 may identify specific portions or sections of a profile to obtain the most relevant information to provide the best version of the OBDI 210 possible for the user. In some embodiments, the relevancy of the information the BIVA 200 retains may be based upon the request of the ODBI 210. For example, if an aspect about a person's reaction after a meeting is requested in the ODBI 210 then only the most relevant information regarding this topic may be selected by the BIVA 200.

In some embodiments, the ODBI 210 may be presented differently based on the relationship between the user who requested versus the user profile being evaluated. In some aspects, the ODBI 210 may be presented differently based on the profile of the user who requested the information. In some implementations, the BIVA 200 may visually highlight the relevant information in the profile of the user to aid with the customized ODBI 210 requested by the user.

In some embodiments, the personal profile may show all non-limiting personal information the user provides, including but not limited to, education history, previous jobs, personality traits psychological evaluations, reviews, and preferences. In some aspects, irrelevant information may be blurred out or not included in the visual representation of the ODBI 210 to keep personal information private from other users.

In some aspects, a clearance level may be needed to access specific information when requesting an ODBI 210. In some implementations, the BIVA 200 may identify the user requesting the ODBI 210 and deny specific information based on relationship, security clearance, relevancy, and other non-limiting factors. For example, if a user requests an ODBI 210 about a personality trait, the BIVA 200 may ignore educational history in the ODBI 210 because of relevancy.

Referring now to FIG. 2B, exemplary process steps for developing and providing ODBI are illustrated. In some embodiments, a BIVA 200 may collect relevant information for an ODBI 210 request regarding a job interview. In some aspects, the BIVA 200 may collect educational information, previous jobs, and other non-limiting factors that may be pulled for relevancy of the ODBI 210. In some implementations, the BIVA 200 may extract specific packages of information or relevant summaries for the ODBI 210 request.

In some aspects, the relevant information may be pulled for different scenarios that may not be related to a job interview. In some implementations, the BIVA 200 may pull relevant information on a case to case basis depending on which information is appropriate for the occasion. For example, the BIVA 200 may pull relevant information for a promotion ODBI 210 request.

In some embodiments, the BIVA 200 may provide the ODBI 210 visually, orally, or both. In some aspects, the BIVA 200 may only provide the ODBI 210 visually to the user via the screen of the device. In some respects, the information may be shown as a list or in different forms for the user to best understand, or the BIVA 100 may show the entire profile of the requested ODBI 210. In some aspects, the BIVA 200 may orally tell the user all of the relevant information in a no specific order.

Referring now to FIG. 2C, exemplary process steps for collecting data that may be used for ODBI are illustrated. In some embodiments, the system 200 may gather data for the user 220 based on tests and different assignments that the user 220 may take, which may be administered by the system or by an external system. In some aspects, the user may be opted into different tests and assignments on a regular basis for the system to collect the most recent and accurate data. In some aspects, the system 200 may prompt users once or periodically to take these tests and assessments. For example, there may be an assignment required once a week and a test required once a month, which may maintain an up-to-date and relevant psychometric profile for each subject.

In some embodiments, individuals within an enterprise or data pool may take different types of tests and assignments. For example, a user 220 may be required to take an assignment about what they most enjoy doing with their free time one week, and the next week they may be required to take a psychological test for evaluation purposes. In some embodiments, each test and assignment may be saved into a personal profile of a specific user 210. In some aspects, the system 200 may autogenerate a summary of each individual test or assignment the user 210 take and put it into their personal profile.

In some embodiments, the system 200 may use the collected information to retain relevant information to return accurate ODBI. In some implementations, based on the collection of data from the system 200, a BIVA may have optimal and accurate information generated in each unique profile for the fastest and most accurate results. In some aspects, the system 200 may automatically update a users' 220 profile once a new test or assignment has been recorded. For example, once a user finishes a test the system 200 may then automatically generate a summary and results to upload to the users' 220 personal profile.

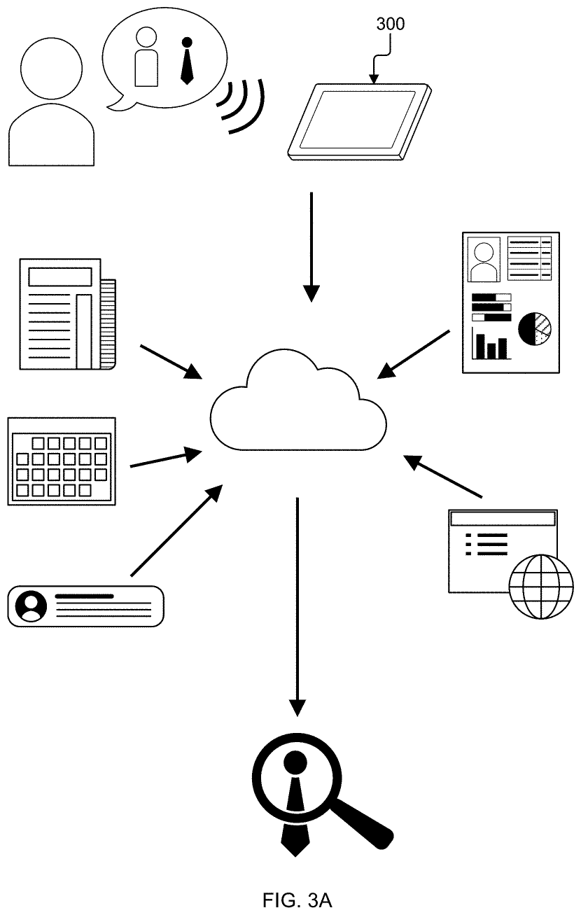

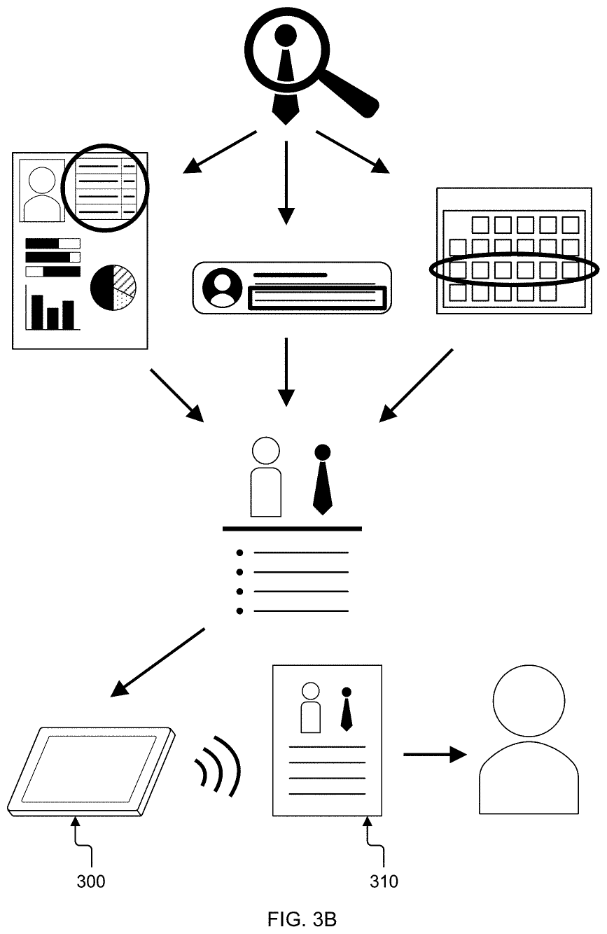

Referring now to FIG. 3A, exemplary sources of information that BIVA may access to develop and provide ODBI are illustrated. In some embodiments, a BIVA 300 may search different resources to gain knowledge about a user based on the ODBI request. In some aspects, a BIVA 300 may search the psychometric profile of the user for an ODBI request. In some implementations, this may be the only resource needed for the ODBI request, and the BIVA 300 may not need to go further in depth with the search.

In some aspects, a requestor may specify which resources to search for ODBI. For example, the requestor may state, “Tell me about Jacob based on his social media” or “Tell me about Sophia based on her psychometric profile.” In some embodiments, a BIVA may selectively access and review profile information. For example, if the request for information about Julia is to prepare for a meeting, a BIVA may deem social media or blog posts as irrelevant. As another example, where a requestor may be looking to connect with the attendees on a personal level, a BIVA may review social media posts and provide ODBI about general topics and preferences based on those posts.

In some embodiments, the BIVA 300 may have access to all content related to the user that the ODBI request has been submitted for. For example, the BIVA 300 may search different content outlets such as, but not limited to: YouTube, Fiverr, personal blog, personal website, Vine, or TikTok. In some aspects, the BIVA 300 may have full access to social media platforms. For example, if an ODBI 310 request requires information related to any misconduct online, the BIVA 300 may then present visual representation of any misconduct or take the user that requested the ODBI directly to the personal social media accounts of the requested user.

In some implementations, a BIVA 300 may have access to a calendar in which the system may keep track of important dates for individual profiles. For example, if the ODBI request asks to recall any birthdays or important milestones of the user the BIVA 300 may have access to that and then summarize all important dates into a ODBI report for the users' request. In some embodiments, if an ODBI request were to ask any general public knowledge about a user the BIVA 300 may have access to articles of the requested user. For example, an ODBI request may be to find if a user has any past history with law enforcement, the BIVA 300 may then research all accessible articles through the system and report back accurate findings based on what law enforcement articles were published, if any. This may not be the only limiting example.

Referring now to FIG. 3B, exemplary extraction steps for identifying relevant data to develop ODBI are illustrated. In some embodiments, a BIVA 300 may recognize a subject requested by the ODBI 310 and summarize any relevant info and report an ODBI 310. In some respects, the BIVA 300 may be directed to a certain subject about the user form the ODBI 310 request, and the BIVA 300 may then identify any relevant information regarding the subject. The BIVA 300 may compile it into a summary and provide an ODBI 310 of the subject matter. For example, the BIVA 300 may be prompted to find any feedback from friends, family, colleagues, or management regarding the user, and once the BIVA 300 has compiled all known knowledge from the system and personal profile of the user then it may provide an ODBI 310 of that subject.

In some aspects, the BIVA 300 may provide summaries of multiple different subject areas at once for the user and compile each subject area into one ODBI 310. For example, the BIVA 300 may be requested to find both previous job history and educational history; the BIVA 300 may then compile all knowledge about both subjects and report back one summarized ODBI 310 to the requestor. In some aspects, the BIVA 300 may have access to all the previously listed resources in FIG. 3A. In some implementations, a BIVA 300 may have limited access to profiles and individual information, and the ODBI 310 may be limited based on that access.

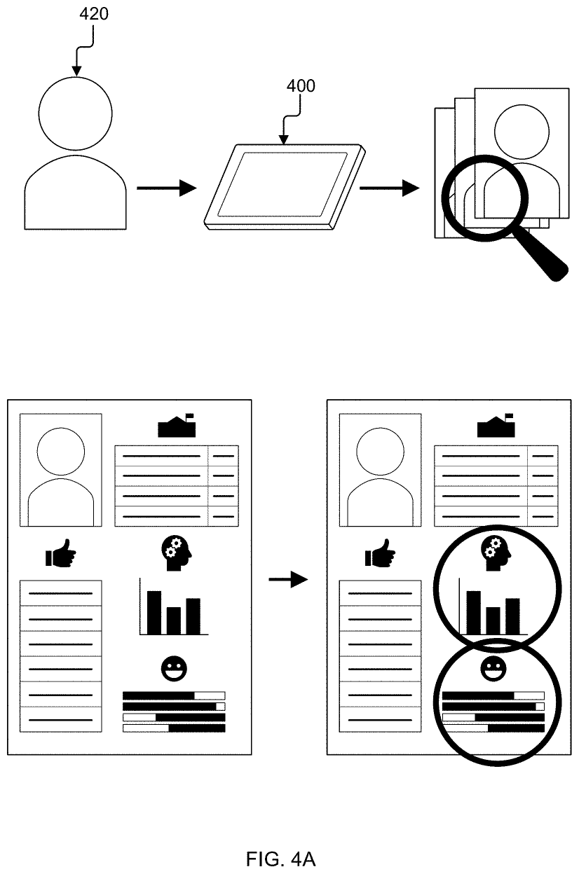

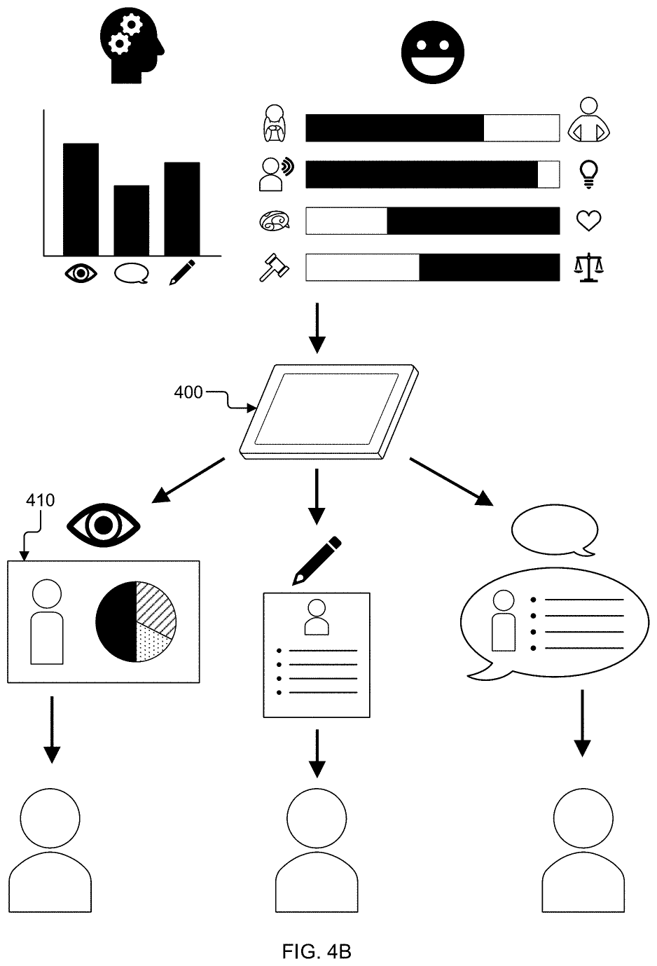

Referring now to FIG. 4A, exemplary process steps for processing a request for ODBI are illustrated, wherein the ODBI may be customized based on behavioral insights of the requestor. In some embodiments, a requestor 420 may request an ODBI, and a BIVA 400 may search for the requestor's 420 profile. A BIVA 400 may extract relevant information and structure the ODBI 410 based at least in part on information related to the requestor 420. In some embodiments, the BIVA 400 may be requested to search a specific profile and provider ODBI with relevant information regarding one specific subject. In some embodiments, the BIVA 400 may be prompted to give general information about a personal profile and then a general summary of that profile may be provided in the ODBI. In some implementations, the BIVA 400 may provide an ODBI of more than one requestor 420 profile at once. For example, if an ODBI is requested for two different subjects regarding two different profiles then the BIVA 400 may fetch the respective information for each requestor 420 profile being requested.

In some respects, the ODBI may be provided visually, orally or both. In some respects, the BIVA 400 may orally summarize the requested information by the requestor 420 in the ODBI. In some embodiments, the ODBI may be visually displayed on the screen for the user 420 to observe, entailing all the requested information regarding the requestor's 420 profile. In some aspects, a visual representation may be present on the screen for the requestor 420 while the BIVA 400 orally summarizes the information regarding the ODBI. In some implementations, BIVA 400 may access the requestor's 420 profile and identify relevant information that may determine how to effectively communicate information to the requestor 420.

Referring now to FIG. 4B, exemplary process steps for customizing ODBI based on behavioral insights of the requestor are illustrated. In some embodiments, BIVA 400 may use different aspects of a user profile to deliver the same information differently in an ODBI 410. In some aspects, based on a specific user requesting an ODBI 410 the information may be delivered orally, visually, or both depending on the user's profile who requested the ODBI 410. In some aspects, a user's profile may indicate that a visual representation of the data may be better suited visually via the ODBI 400. In some implementations, a BIVA 400 may recognize that the user requesting the ODBI 410 may use an oral representation of the data batter rather than a visual. In some embodiments, the BIVA 400 may recognize that a user relates to the information better transmitted both visually and orally and the ODBI 410 may be presented that way.

In some embodiments, the BIVA 400 may optimize relevant information to a user that requested the ODBI 410, which may allow the user to process the information effectively. In some implementations, the BIVA 400 may use a user's psychometric profile to assess which order the relevant information may need to come in at for the user to properly understand it through the ODBI 410. In some aspects, the BIVA 400 may analyze how the ODBI 410 has been requested by the user and feed information to the user via a specifically designed informational ODBI 410 based on the users profile and wording of the request.

In some respects, the BIVA 400 may recognize a frequent user's voice and automatically pull their profile and cater the ODBI 410 to the specific user who is requesting the ODBI 410. In some embodiments, a user may need to sign into the system and the BIVA 400 may recognize the profile and cater all ODBI 410 requests toward the particular profile of the user requesting the ODBI 410.

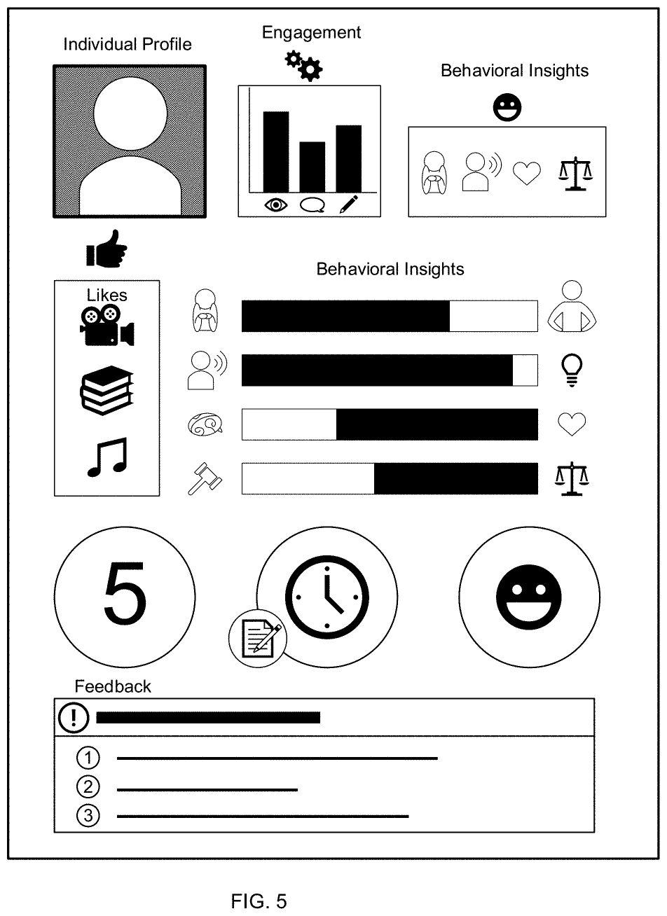

Referring now to FIG. 5, an exemplary behavioral insight dashboard for an individual is illustrated. In some aspects, behavioral insights may be presented as a combination of graphics, numbers, and text. In some embodiments, the behavioral insights may be presented in context of employment, wherein information is provided for both the individual and for those who may work with the individual.

In some implementations, the behavioral insight dashboard may present success predictions for various roles, such as within different company sizes, company stages, and management positions, as non-limiting examples. In some embodiments, the behavioral insight dashboard may logically interface with third party psychometric assessment platforms, wherein the behavioral insights may be based in part on data associated with the individual from the platforms. In some aspects, the behavioral insight dashboard may incorporate other attributes, such as interests, skills, aspirations, experience, performance data, and feedback data, as non-limiting examples.

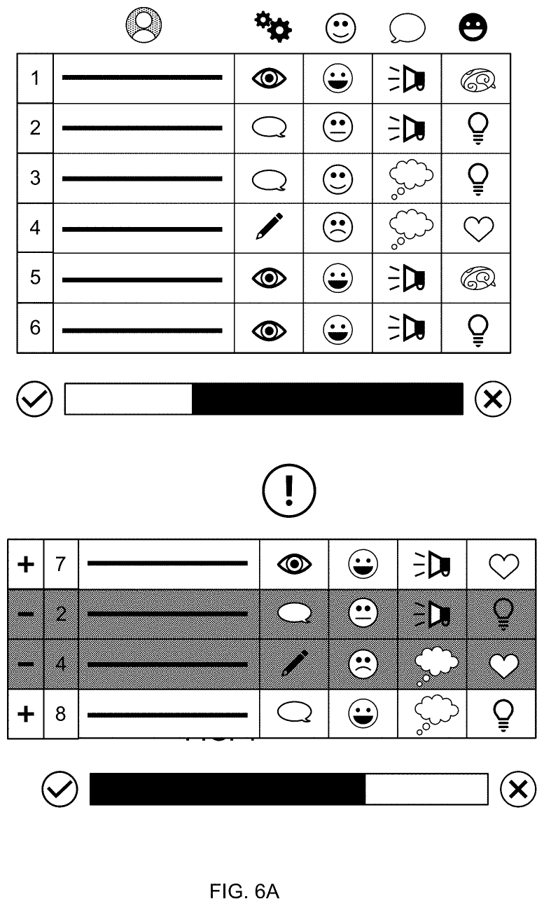

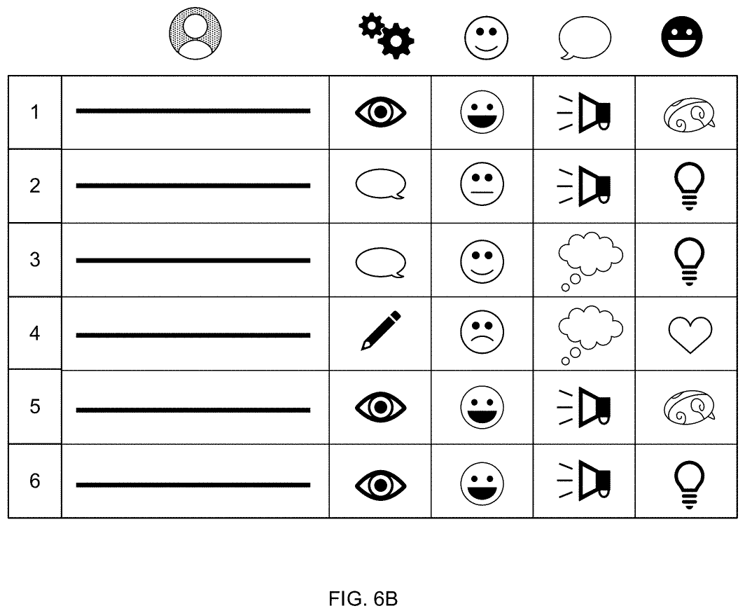

Referring now to FIG. 6A, an exemplary team behavioral insight dashboard is illustrated. In some aspects, the aggregated psychometric profiles and behavioral insights may be provided for a team. In some embodiments, a team may be manually selected or may be based on identified groups, such as by project, department, position, location, or performance types, as non-limiting examples.

In some implementations, it may be useful to view the overall makeup of a team to understand how they can be successful. For example, knowing that a team is lacking any member with an attention to detail may prompt the company to add a detail-oriented person to the team who may complement the other team attributes. As another example, knowing that a team is competitive in a productive manner may inform how projects are presented to them so as to encourage their competitive nature.

In some aspects, as team members may be added and removed, the aggregate behavioral insights may change to reflect the removed or added psychometric profiles. In some embodiments, this may occur in real time. This may allow for immediate behavioral insights based on adjustments to team dynamics. A team builder may utilize this feature to tailor a team to a specific goal. In some aspects, a team builder may utilize this feature to adjust a team to be more effective.

In some embodiments, the system may suggest ways to improve a team or may suggest team memberships. For example, based on behavioral insights, the system may suggest team building exercises that may increase camaraderie for one team, and the system may suggest adding in a detail-oriented member to a team that lacks that attribute. In some aspects, behavioral insights may allow a company to develop an effective team for a specific client, for a specific task, for a specific department, or for a specific role, as non-limiting examples.

Referring now to FIG. 6B, exemplary behavioral insights for a team are illustrated, wherein the behavioral insights are based at least in part on psychometric profiles. In some embodiments, a team may comprise an organizational team, such as within a particular department or assigned to a particular project. In some aspects, a team may comprise an invitee list for a meeting, as the invitees may be considered a specialty team assigned to the goal or purpose of the meeting.

In some implementations, a team may comprise the founders of a company or the c-suite of a company. In some aspects, a team may be an existing team or one that has not yet been built. Understanding the aggregate behavioral insights and psychometric profiles of a team may allow for a better understanding on how to help the team succeed. It may be helpful to anticipate the strengths and weaknesses of a team before it is created. In some aspects, the team may be adjusted to create a more balanced or effective aggregate psychometric profile.

For example, a venture capital group may be interested in investing in a startup with three founders. An understanding of their behavioral insights and psychometric profiles, both aggregated and individual, may allow them to make a more informed decision. They may use the information to add or remove team members to increase the chance of success of the startup. In some embodiments, there may be known psychometric profiles or identified successful teams for particular goals, such as founders of a successful startup, wherein a comparison of a team's psychometric profiles and behavioral insights may be useful to predict outcomes.

In some aspects, a team report dashboard may present available team reports, wherein clicking into each team label may link to each individual team report. In some embodiments, the employee list may indicate which employees have linked their psychometric profiles to the organization. For employees who have not linked their psychometric profiles, the team report dashboard may provide an option to send a request, which may prompt the employee to link their psychometric profile. If the employee has not yet created a psychometric profile, the request may prompt them to create one.

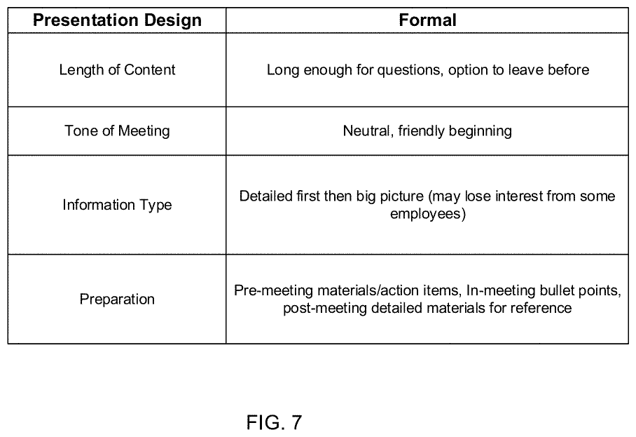

Referring now to FIG. 7, exemplary behavioral insights and meeting strategy suggestions are illustrated. In some aspects, a summary of behavioral insights for invitees may be provided, which may allow the host to prepare based on the summary. In some embodiments, the summary may be paired with more detailed behavioral insights. In some implementations, the summary may be based on the percent of invitees with particular psychometric profile attributes, wherein the summary indicates behavioral insights for the majority of the invitees.

In some embodiments, the summary may be based on the most sensitive psychometric profile attributes. For example, eight out of ten invitees may benefit from a big picture presentation, and two out of ten invitees could not be effective without the details. Those eight out of ten may not be negatively impacted by the details, so the summary may suggest the presentation of details. A system providing a dynamic summary of behavioral insights may process the psychometric profiles of each invitee and weigh their attributes and behavioral insights, wherein the summary may provide group behavioral insights and suggested meeting strategies based on weighted assessments.

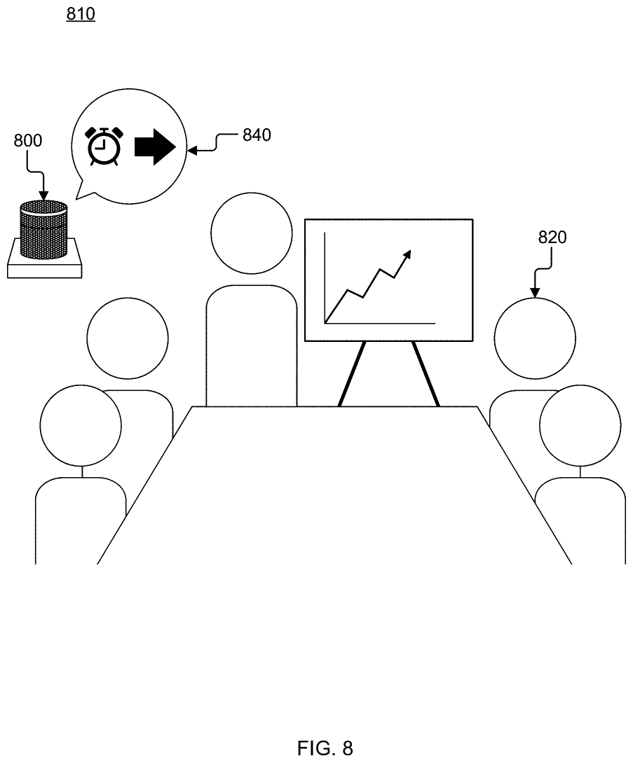

Referring now to FIG. 8, exemplary ODBI 810 is illustrated. In some embodiments, the ODBI 810 may provide behavioral insights to a plurality of users 820. In some implementations, the BIVA 800 may provide ODBI 810 autonomously. In some aspects, the ODBI 810 may be provided based on predetermined external cues.

As an illustrative example, the BIVA 800 may be placed within a meeting. The BIVA 800 may utilize machine learning to recognize conversational lulls within the meeting. The BIVA 800 may then provide a non-verbal ODBI 810 is the form an audible notification. The audible notification 840 may indicate to the presenter that it is time to change discussion topics within the meeting.

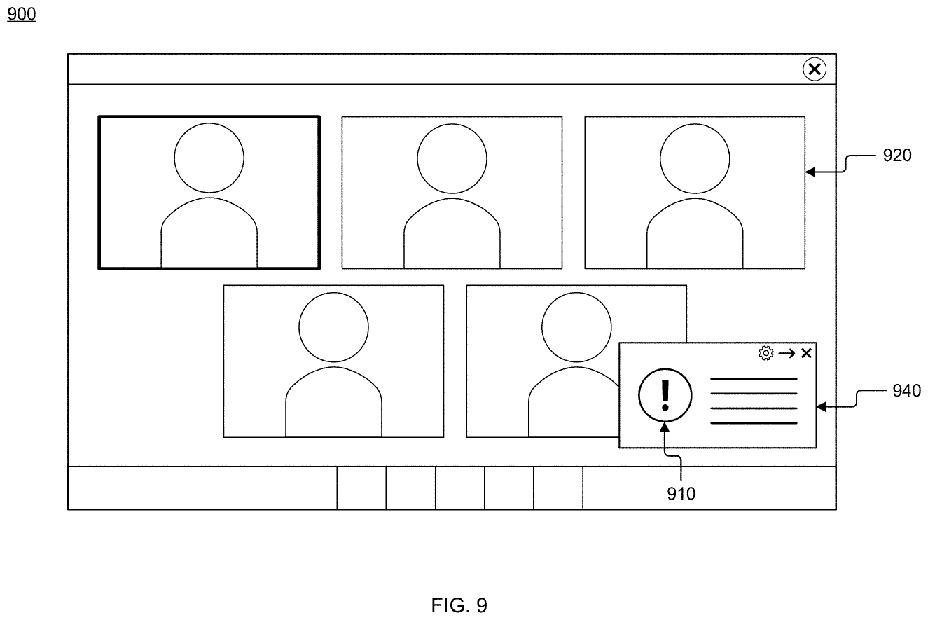

Referring now to FIG. 9, exemplary BIVA 900 is illustrated. In some embodiments, the BIVA 900 may provide personalized notifications 940 based on behavioral insights. As an example, the BIVA 900 may be present as a monitoring software within a virtual meeting. The BIVA 900 may be cognizant of which users 920 are present within a meeting. The BIVA may utilize machine learning to provide personalized notifications 940 to specific users 920 during the meeting based on their behavioral insights. The BIVA 900 may access the user-facing camera to learn that a user 920 tends to lose focus at a certain time duration within meetings. The BIVA 900 may send a personal notification to the user 920 at this machine-learned time interval to increase attentiveness for the user 920.

In some embodiments, the BIVA 900 may utilize behavioral insights to interact with a plurality of users 920 simultaneously. In some implementations, the BIVA 900 may provide ODBI 910 autonomously. In some aspects, the BIVA 900 may provided ODBI 910 based on predetermined external cues. As an illustrative example, the BIVA 900 may be placed within a meeting. The BIVA 900 may utilize machine learning to recognize conversational lulls within the meeting. The BIVA 900 may then provide a non-verbal ODBI 910 is the form an audible notification. The audible notification 940 may indicate to the presenter that it is time to change discussion topics within the meeting.

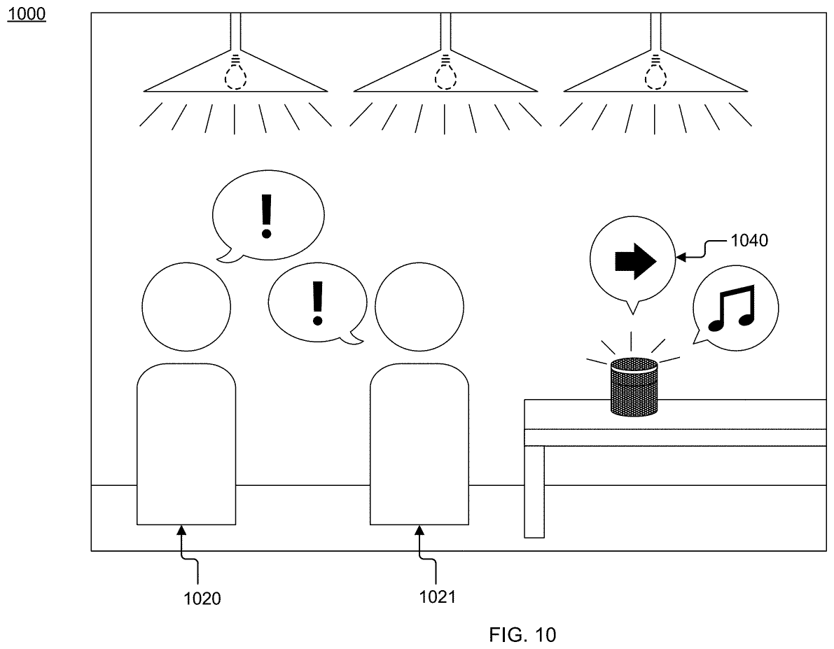

Referring now to FIG. 10, exemplary BIVA 1000 is illustrated. In some embodiments, the BIVA 1000 may react to environmental cues. For example, two users 1020, 1021 may converse in proximity to a BIVA 1000 that may actively monitor its surroundings. The users 1020, 1021 may begin to become angry in the midst of the discussion. The BIVA 1000 may detect anomalies in the tone of the users 1020, 1021 and engage the environment in methods that may induce a calming effect on the users 1020, 1021. The BIVA 1000 may dim the lights in the room and play quiet music in the background to reduce tension between the users 1020, 1021. The BIVA 1000 may provide auditory suggestions 1040 for resolution by providing ODBI 1010 based on the behavioral insights of the users 1020, 1021.

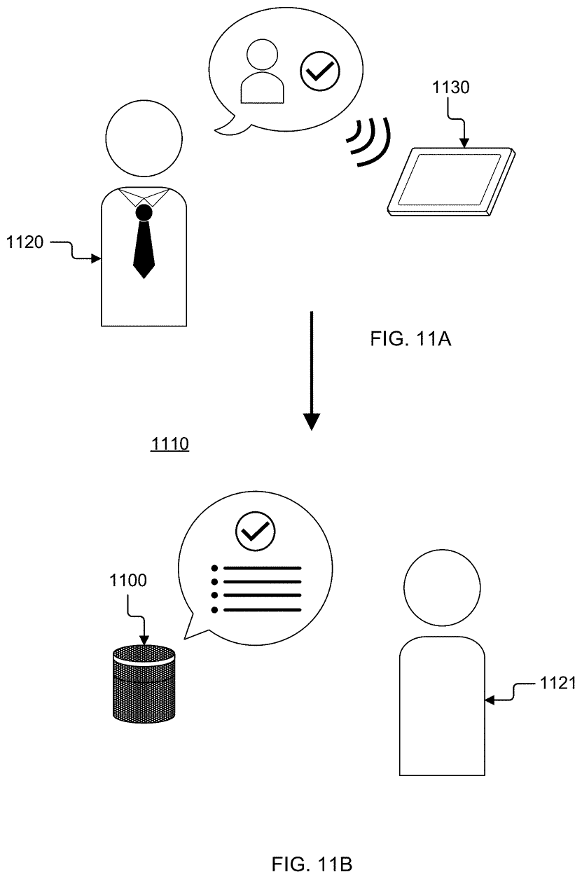

Referring now to FIGS. 11A-11B, an exemplary ODBI 1110 for a user is illustrated. In some embodiments, the OBDI 1110 may be sent from a user 1120. In some implementations, the ODBI 1110 may be communicated via the BIVA 1100 to a user 1121. As an illustrative example, an employer may conduct a quarterly assessment with each employee. The employer may dictate observations to add to the employee's record. The behavioral insight profile may adjust based upon the employers feedback. The BIVA 1100 may announce an update to the employee's behavioral insight profile to the employee. A summary of the feedback provided and the behavioral insight profile may be presented to the employee.

In some embodiments, the BIVA 1100 may utilize a computer-implemented method for providing on-demand behavioral insights, wherein on-demand behavioral insights may be provided through the BIVA 1100. In some implementations, the BIVA 1100 may receive an activation prompt requesting logical access to the behavioral insights of a subject. In some aspects, the activation prompt may be received through the BIVA. In some embodiments, the BIVA 1100 may receive a communication destination.

In some implementations, the BIVA 1100 may receive an identifier of the subject. In some aspects, the BIVA 1100 may receive a purpose for accessing behavioral insights of the subject. In some embodiments, the BIVA 1100 may access a subject profile through at least one database. In some aspects, the subject profile may comprises at least the identifier and behavioral insights of the subject. In some embodiments, the BIVA 1100 may generate on-demand behavioral insights, wherein the on-demand behavioral insights are based on predefined threshold parameters comparing the purpose to the subject profile. In some implementations, the BIVA 1100 may communicate on-demand behavioral insights to the communication destination.

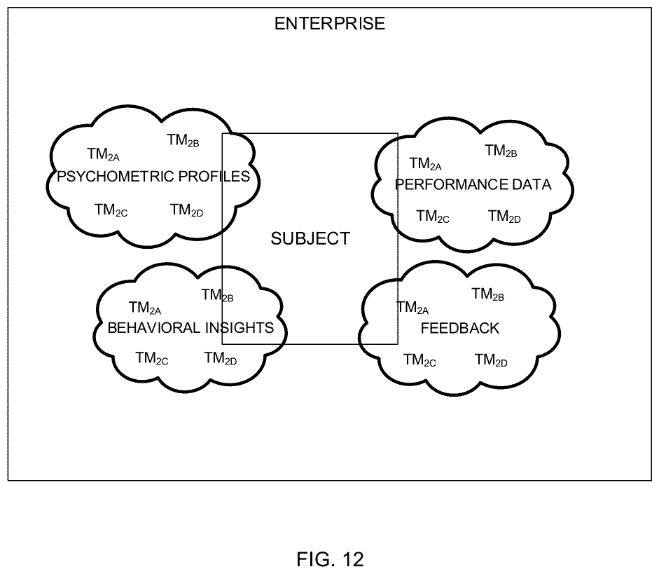

Referring now to FIG. 12, exemplary distribution of behavioral insights within an organization is illustrated. In some embodiments, an organization may maintain databases with psychometric profiles, performance data, behavioral insights, and feedback. In some aspects, these databases may be maintained in groups or separately. In some implementations, the databases may be organized or categorized by subject, such as by person, team, or position, as non-limiting examples. In some aspects, the databases may be logically linked, which may allow for correlation between the data, such as by subject.

For example, the subject may comprise an individual, and her psychometric profile and behavioral insights may be compared to her performance and feedback. This may allow for an assessment of whether she is performing above or below expectations based on her behavioral insights. In some aspects, her performance may be compared to her feedback, which may provide insight as to how effective the feedback has been and whether her responses have been in line with expectations or predictions based on her psychometric profile and behavioral insight.

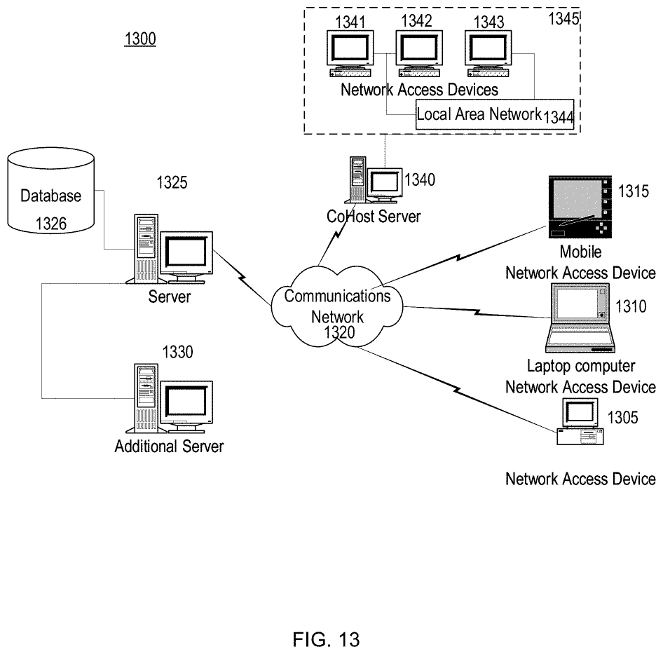

Referring now to FIG. 13, an exemplary processing and interface system 1300 is illustrated. In some aspects, access devices 1315, 1310, 1305, such as a paired portable device 1315 or laptop computer 1310 may be able to communicate with an external server 1325 though a communications network 1320. The external server 1325 may be in logical communication with a database 1326, which may comprise data related to identification information and associated profile information. In some embodiments, the server 1325 may be in logical communication with an additional server 1330, which may comprise supplemental processing capabilities.

In some aspects, the server 1325 and access devices 1305, 1310, 1315 may be able to communicate with a cohost server 1340 through a communications network 1320. The cohost server 1340 may be in logical communication with an internal network 1345 comprising network access devices 1341, 1342, 1343 and a local area network 1344. For example, the cohost server 1340 may comprise a payment service, such as PayPal or a social network, such as Facebook or a LinkedIn.

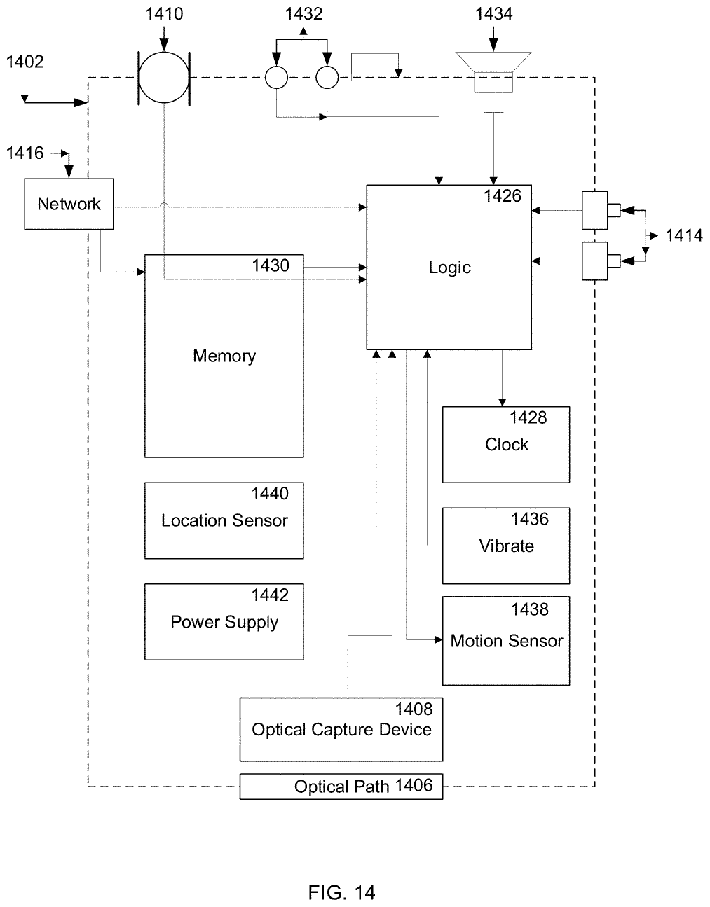

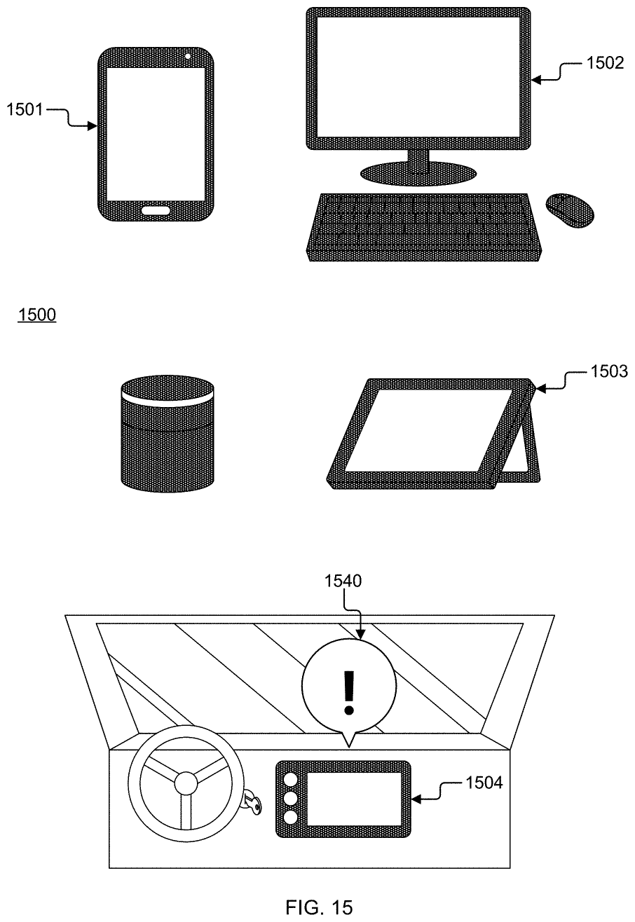

Referring now to FIG. 14, an exemplary block diagram of an exemplary embodiment of a mobile device 1402 is illustrated. The mobile device 1402 may comprise an optical capture device 1408, which may capture an image and convert it to machine-compatible data, and an optical path 1406, typically a lens, an aperture, or an image conduit to convey the image from the rendered document to the optical capture device 1408. The optical capture device 1408 may incorporate a Charge-Coupled Device (CCD), a Complementary Metal Oxide Semiconductor (CMOS) imaging device, or an optical sensor of another type.

In some embodiments, the mobile device 1402 may comprise a microphone 1410, wherein the microphone 1410 and associated circuitry may convert the sound of the environment, including spoken words, into machine-compatible signals. Input facilities 1414 may exist in the form of buttons, scroll-wheels, or other tactile sensors such as touchpads. In some embodiments, input facilities 1414 may include a touchscreen display. Visual feedback 1432 to the user may occur through a visual display, touchscreen display, or indicator lights. Audible feedback 1434 may be transmitted through a loudspeaker or other audio transducer. Tactile feedback may be provided through a vibration module 1436.

In some aspects, the mobile device 1402 may comprise a motion sensor 1438, wherein the motion sensor 1438 and associated circuity may convert the motion of the mobile device 1402 into machine-compatible signals. For example, the motion sensor 1438 may comprise an accelerometer, which may be used to sense measurable physical acceleration, orientation, vibration, and other movements. In some embodiments, the motion sensor 1438 may comprise a gyroscope or other device to sense different motions.

In some implementations, the mobile device 1402 may comprise a location sensor 1440, wherein the location sensor 1440 and associated circuitry may be used to determine the location of the device. The location sensor 1440 may detect Global Position System (GPS) radio signals from satellites or may use assisted GPS where the mobile device may use a cellular network to decrease the time necessary to determine location. In some embodiments, the location sensor 1440 may use radio waves to determine the distance from known radio sources such as cellular towers to determine the location of the mobile device 1402. In some embodiments these radio signals may be used in addition to and/or in conjunction with GPS.

In some aspects, the mobile device 1402 may comprise a logic module 1426, which may place the components of the mobile device 1402 into electrical and logical communication. The electrical and logical communication may allow the components to interact. Accordingly, in some embodiments, the received signals from the components may be processed into different formats and/or interpretations to allow for the logical communication. The logic module 1426 may be operable to read and write data and program instructions stored in associated storage 1430, such as RAM, ROM, flash, or other suitable memory. In some aspects, the logic module 1426 may read a time signal from the clock unit 1428. In some embodiments, the mobile device 1402 may comprise an on-board power supply 1442. In some embodiments, the mobile device 1402 may be powered from a tethered connection to another device, such as a Universal Serial Bus (USB) connection.

In some implementations, the mobile device 1402 may comprise a network interface 1416, which may allow the mobile device 1402 to communicate and/or receive data to a network and/or an associated computing device. The network interface 1416 may provide two-way data communication. For example, the network interface 1416 may operate according to an internet protocol. As another example, the network interface 1416 may comprise a local area network (LAN) card, which may allow a data communication connection to a compatible LAN. As another example, the network interface 1416 may comprise a cellular antenna and associated circuitry, which may allow the mobile device to communicate over standard wireless data communication networks. In some implementations, the network interface 1416 may comprise a Universal Serial Bus (USB) to supply power or transmit data. In some embodiments, other wireless links known to those skilled in the art may be implemented.

Referring now to FIG. 15, a plurality of exemplary BIVA 1500, 1501, 1502, 1503, 1504 are illustrated. In some embodiments, the BIVA 1502, 1503 may comprise a software. In some implementations, the BIVA 1502, 1503 may be installed on a plurality of devices. For example, the BIVA 1502, 1503 may be downloadable software for laptops and desktops and share information between devices. A mobile application may share the same information as the desktop and the laptop.

In some embodiments, a BIVA 1501, 1504 may comprise a mobile application. In some implementations, the BIVA 1501, 1504 may interface with a plurality of mobile interfaces. In some aspects, the BIVA 1501, 1504 may interface with a plurality of mobile platforms. In some embodiments, the BIVA 1503 may present visual representations of ODBI on passive devices. For example, a BIVA 1500 may receive a verbal request for an ODBI that may display on a separate BIVA 1503.

In some embodiments, the BIVA 1504 may provide notifications 1540 in response to an external stimulus. As an illustrative example, the BIVA 1504 may be a software loaded into the computer components of a vehicle. The BIVA 1504 may provide notifications 1540 that assist the driver with alertness based upon a behavioral insight profile.

In some embodiments, the BIVA 1500 may utilize a computer-implemented method for providing on-demand behavioral insights, wherein on-demand behavioral insights may be provided through the BIVA 1500. In some implementations, the BIVA 1500 may receive an activation prompt requesting logical access to the behavioral insights of a subject. In some aspects, the activation prompt may be received through the BIVA. In some embodiments, the BIVA 1500 may receive a communication destination.

In some implementations, the BIVA 1500 may receive an identifier of the subject. In some aspects, the BIVA 1500 may receive a purpose for accessing behavioral insights of the subject. In some embodiments, the BIVA 1500 may access a subject profile through at least one database. In some aspects, the subject profile may comprises at least the identifier and behavioral insights of the subject. In some embodiments, the BIVA 1500 may generate on-demand behavioral insights, wherein the on-demand behavioral insights are based on predefined threshold parameters comparing the purpose to the subject profile. In some implementations, the BIVA 1500 may communicate on-demand behavioral insights to the communication destination.

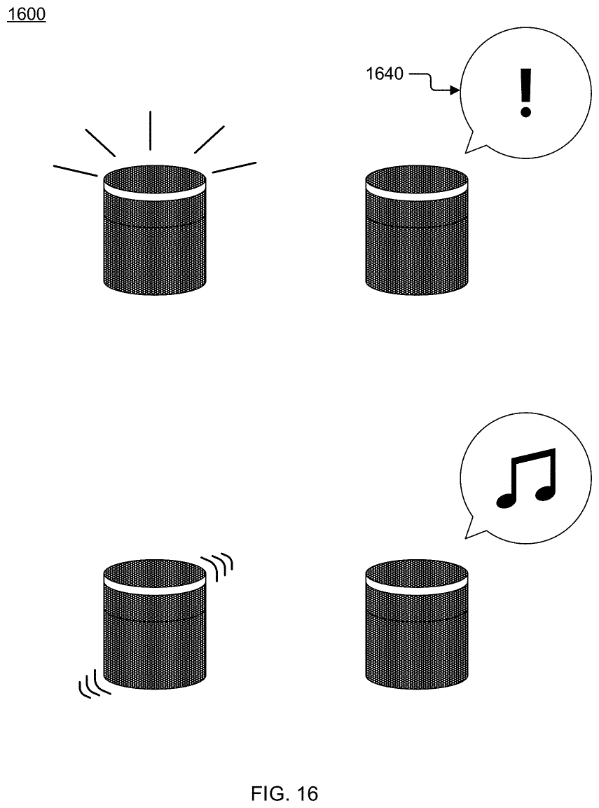

Referring now to FIG. 16, an exemplary BIVA 1600 with a plurality of notification 1640 types is illustrated. In some embodiments, the BIVA 1600 may provide an audible response. In some implementations, the BIVA 1600 may emit a nonverbal audio notification 1640 similar to a ding, as a non-limiting example. In some aspects, the BIVA 1600 may emit a verbal notification 1640 that indicates the reason for the notification 1640. For example, the BIVA 1600 may audibly describe a user's behavioral insight profile upon request.

In some embodiments, the BIVA 1600 may provide a visual notification 1640. For example, the BIVA 1600 may be activated audibly by a specific phrase and the BIVA 1600 may indicate a ready state for instruction by illuminating green. In some implementations, the BIVA 1600 may provide haptic notifications 1640. For example, a phone may comprise BIVA 1600 as a loaded app that is downloading a behavioral insights profile. The BIVA 1600 may cause the phone to vibrate to notify the user that the download is complete.

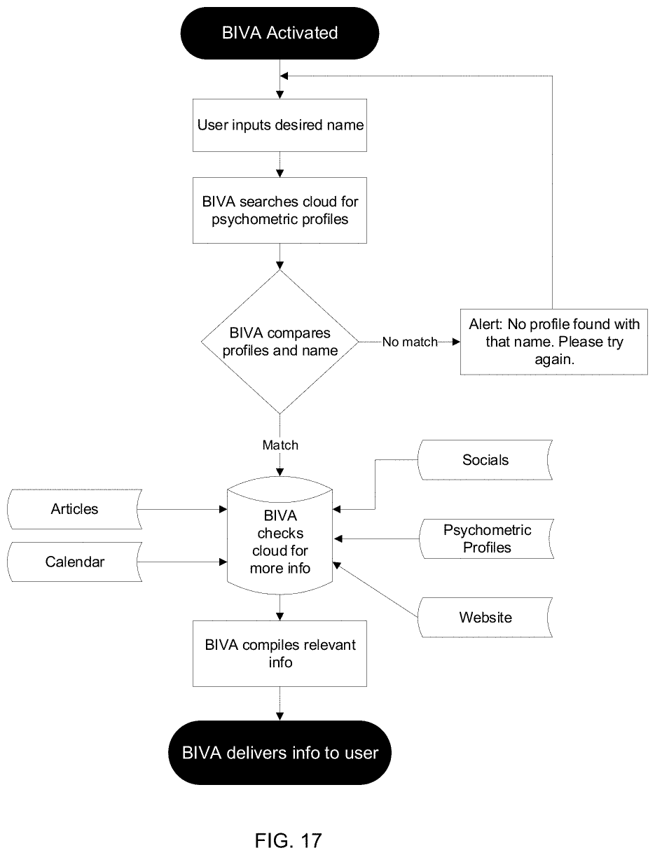

Referring now to FIG. 17, an exemplary flowchart for collecting and delivering ODBI are illustrated. In some embodiments, an exemplary flowchart may illustrate how a BIVA may use the system to take relevant information and deliver the ODBI. BIVA may be activated, and the user may input a person or persons name into the system, such as through text or orally. In some aspects, the user may request more than one user at a time and the BIVA may pull information about all requested parties. In some embodiments, the user may input specific information required for the ODBI. In some implementations, the user may request different types of information from different profiles, and the BIVA may select which information is relevant for each profile. For example, a user may request education history from one profile, and job history from another profile and the BIVA may extract the proper information for the ODBI.

In some implementations, the system may check for existing profiles based on at least a portion of the information and names of the profiles have been inputted. In some aspects, a profiles may not exist for the requested person, which may prompt the requestor to repeat the name. If no profile exists, the system may send a prompt to the requested individual to initiate a profile, such as by prompting them to complete a psychometric profile. In some respects, the names may match existing profiles and the system may continue with the process. In some implementations, if there exists more than one profile of the same name, the user may be asked different questions to help better identify from which profile the information is needed. For example, the user may be asked to specify the gender, age, position, or employer, as non-limiting examples.

The correct profile information may be identified, and the system may check the cloud for more information regarding the profile. The cloud may have information from other resources, such as previously mentioned in FIG. 3A. In some aspects, based on the information based on the requested information by the user, the BIVA may compile relevant information regarding the information needed. Once all the relevant information is collected by the BIVA, the ODBI may be delivered to the user.

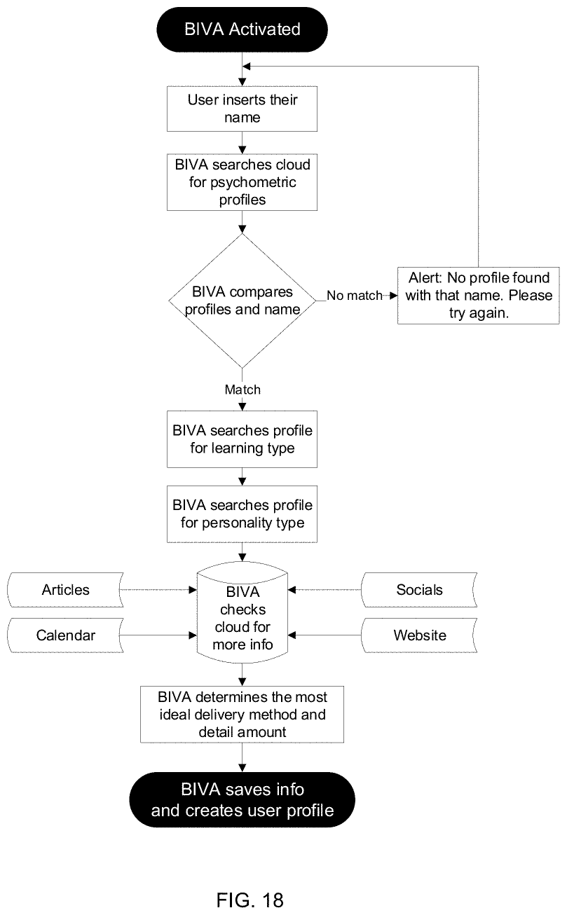

Referring now to FIG. 18, an exemplary flowchart for collecting and delivering ODBI is illustrated. In some embodiments, the flowchart may illustrate how a user may create and setup a personal profile. In some aspects, BIVA may need to be activated before the device allows for the user to setup a profile. Once BIVA is activated the user may be prompted to input the name of the personal profile that may be shown when an ODBI is requested. For example, the user may input their personal name when prompted and that name may be displayed to all users across the platform. Once the name has been setup the system may cross reference the name with a pre-existing psychometric profile already registered in the system. In some aspects, the system may not find a pre-existing psychometric profile and the user may then need to create one before they may proceed.

In some respects, multiple psychometric profiles may exist with the same name, and the user may need to provide more information for the system to identify the correct psychometric profile for the correct profile. Once a match for the profiles has been found for the system may then search for a learning and personality type of that user. In some aspects, different users may benefit from different types of information delivery styles. For example, one user may learn more visually than another or more orally than another user, and the system may determine the category based in part on the behavioral insights associated with the person requesting ODBI.

Once the system has categorized the user appropriately, it may search the cloud for more information regarding the user. In some aspects, the system may use outside resources, such as previously mentioned in FIG. 3A, to supplement psychometric profile information and to refine the user's profile. In some respects, the cloud may automatically update the user's profile once new information has been discovered. Once the cloud has finished updating the user's profile, a BIVA may determine the most ideal delivery method for the ODBI to the user. Based on that determination, BIVA may provide the ODBI by an effective delivery method to the user.

In some aspects, requests for ODBI may be collected and stored, such as with the subject profile, the requestor's profile, or with the enterprise database. A record of ODBI requests may provide insights as to how many people engage with the subject or how often a requestor works to incorporate behavioral insights into their interactions. Across an enterprise, a record may indicate how actively their employee pool engages with each other on a behavioral level.

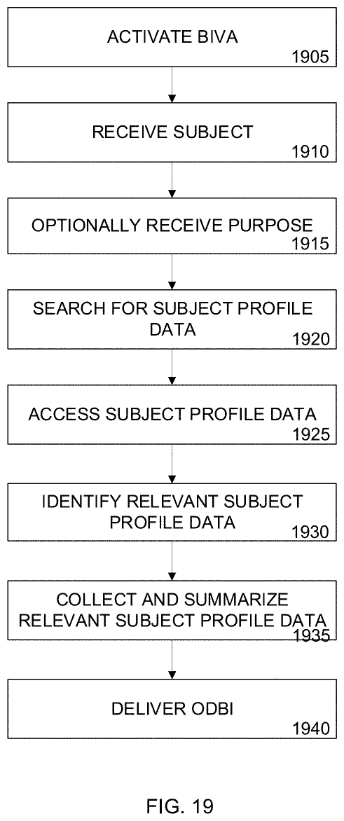

Referring now to FIG. 19, exemplary method steps for receiving an ODBI request and providing an ODBI are illustrated. At 1905, a BIVA may be activated, such as by speaking an activation word or by digitally engaging through a device. At 1910, a subject may be received. In some aspects, at 1915, a purpose may be received, such as for a meeting, for an interview, or for a date. At 1920, subject profile data may be researched. At 1925, subject profile data may be accessed. At 1930, relevant subject profile data may be identified. At 1935, relevant subject profile data may be collected and summarized. At 1940, ODBI may be delivered.

In some embodiments, BIVA may process an ODBI using the BIVA system. In some aspects, BIVA may need to be activated, either by the user manually or BIVA may automatically activate by voice recognition or motion sensing, but these may not be the only limiting examples. Once BIVA is activated it may then receive the name of the profile the ODBI is being requested from. After the correct name is received the user may the request an event from the user that may relate to the profile requested. In some implementations, the event may be specific information requested in an ODBI related to the name of the profile. Once the event and name have been identified from BIVA, the cloud system may then identify the correct profile and info related to the ODBI requested.

Once BIVA has correctly identified the profile and information it may check this profile for a relevant information regarding the ODBI requested by the user. After the profile has been identified and reviewed for relevant information BIVA may then compile any and all relevant information regarding the requested ODBI. Finally, after the proper information and profile have been gathered by BIVA then an ODBI delivery may be delivered by BIVA. In some aspects, the ODBI may be delivered differently for different user based on their psychometric profile and learning categories.

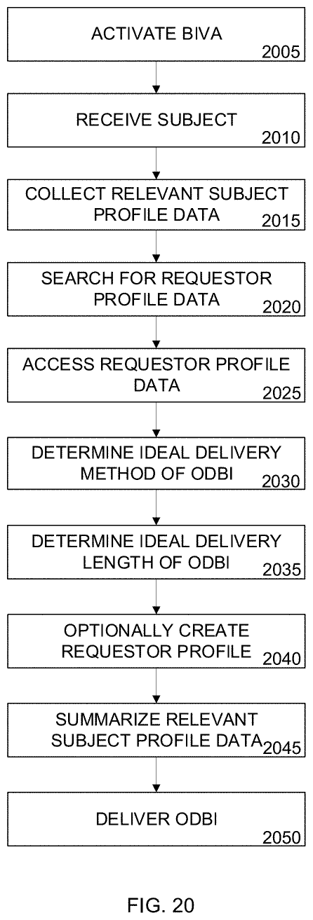

Referring now to FIG. 20, exemplary methods steps for receiving an ODBI request and providing an ODBI are illustrated. At 2005, BIVA may be activated. At 2010, a subject may be received. At 2015, relevant subject profile data may be collected. At 2020, requestor profile data may be researched. At 2025, requestor profile data may be accessed. At 2030, an ideal delivery method of ODBI may be determined. At 2035, an ideal delivery length of ODBI may be determined. In some aspects, at 2040, a requestor profile may be created. At 2045, relevant subject profile data may be summarized. At 2050, ODBI may be delivered.

In some embodiments, BIVA may view the process of creating a user profile. Firstly, activation of BIVA may be required to start the process. In some aspects, BIVA may automatically activate or may be manually activated by the user on the system. Once BIVA has been activated it may receive a username created and given to the system by the user. After the username has been cleared by the system BIVA may search the cloud for the person creating the profile, identifying their profile and information from the cloud system. In some respects, there may exist more than one person with similar names and the BIVA may require the user to answer further question to narrow down the specific user creating the profile.

Once the profile and information have been properly identified by BIVA it may check the profile for information regarding how to properly categorize the user's learning and personality types. In some respects, based on specific information and profile type BIVA may categorize a user in specific categories for each learning and personality type. After BIVA has properly categorized the user in their perspective categories it may determine the best way to deliver ODBI to that particular user. Once the user has been categorized BIVA may then determine how much detail each user may be required to have in their profile and in their ODBI requests.

In some aspects, BIVA may compile all the previous information and save it into the system creating the profile for the user. In some aspects, each profile may be different based on information and information provided by the user, BIVA may categorize each user differently. In some embodiments, once the profile has been created it may automatically update each time BIVA receives new information regarding the user and their profile. In some embodiments, BIVA may change the categorization of the user based new information provided or discovered by BIVA.

### Conclusion

A number of embodiments of the present disclosure have been described. While this specification contains many specific implementation details, there should not be construed as limitations on the scope of any disclosures or of what may be claimed, but rather as descriptions of features specific to particular embodiments of the present disclosure.

Certain features that are described in this specification in the context of separate embodiments can be implemented in combination in a single embodiment. Conversely, various features that are described in the context of a single embodiment can be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. In certain implementations, multitasking and parallel processing may be advantageous. Nevertheless, it will be understood that various modifications may be made without departing from the spirit and scope of the claimed disclosure.

What is claimed is:

1. A computer-implemented method for providing on-demand behavioral insights, wherein on-demand behavioral insights are provided through a behavioral insight virtual assistant, the computer-implemented method comprising:
receiving an activation prompt requesting logical access to behavioral insights of a subject, wherein the activation prompt is received through a behavioral insight virtual assistant;
receiving a communication destination configured to deliver on-demand behavioral insights;
receiving an identifier of the subject;
receiving a purpose for accessing behavioral insights of the subject;
accessing a subject profile through at least one database, wherein the subject profile comprises at least the identifier and behavioral insights of the subject;
generating on-demand behavioral insights, wherein on-demand behavioral insights are based on predefined threshold parameters comparing the purpose to the subject profile; and
communicating on-demand behavioral insights to the communication destination.

2. The computer-implemented method of claim 1, wherein behavioral insights of the subject comprise a plurality of behavioral insight types, wherein the predefined threshold parameters comprise at least one requested behavioral insight type from the plurality of behavioral insight types.

3. The computer-implemented method of claim 1, wherein the purpose comprises conducting a meeting and the subject comprises an attendee of the meeting.

4. The computer-implemented method of claim 3, wherein on-demand behavioral insights relate to information on how to effectively conduct the meeting for the subject.

5. The computer-implemented method of claim 1, wherein the subject comprises a plurality of individuals.

6. The computer-implemented method of claim 5, wherein behavioral insights of the subject comprise an aggregation of behavioral insights of the plurality of individuals.

7. The computer-implemented method of claim 5, wherein behavioral insights of the subject comprise a summary of behavioral insights of the plurality of individuals.

8. The computer-implemented method of claim 1, wherein on-demand behavioral insights are provided based on predefined format requirements.

9. The computer-implemented method of claim 8 further comprising:
receiving a requestor profile comprising behavioral insights of a requestor of the activation prompt, wherein predefined format requirements are based at least in part on behavioral insights associated with the requestor.

10. The computer-implemented method of claim 8, further comprising:
assigning a weighted value to behavioral insights of the subject, wherein predefined threshold parameters are based on the weighted value, and wherein the format is based at least in part on the weighted value.

11. A system for providing on-demand behavioral insights, the system comprising:
a behavioral insight virtual assistant configured to receive on-demand behavioral insight requests and to provide on-demand behavioral insights;
a behavioral insight server in wireless communication with the behavioral insight virtual assistant;
a plurality of databases comprising two or more of:
a psychometric profile database,
a skills database,
a personal information database,
an executable software, wherein the executable software instructs the behavioral insights server to:
receive an activation prompt requesting logical access to behavioral insights of a subject, wherein the activation prompt is received through the behavioral insight virtual assistant;
receive a communication destination to deliver on-demand behavioral insights;
receive an identifier of the subject;
receive a purpose for accessing behavioral insights of the subject;
receive a requestor profile through at least one database;
access a subject profile through at least one of the plurality of databases, wherein the subject profile comprises at least the identifier and behavioral insights of the subject;
generate on-demand behavioral insights, wherein the on-demand behavioral insights are based on predefined threshold parameters comparing the purpose to the subject profile; and
communicate on-demand behavioral insights to the communication destination.

12. The system of claim 11, wherein requesting logical access to behavioral insights is received as audio.

13. The system of claim 11, wherein the behavioral insight virtual assistant comprises the communication destination.

14. The system of claim 11, wherein on-demand behavioral insights are provided based on predefined format requirements.

15. The system of claim 14, wherein the executable software instructs the behavioral insight server to:
receive a requestor profile comprising behavioral insights of a requestor of the activation prompt, wherein predefined format requirements are based at least in part on behavioral insights associated with the requestor.

16. The system of claim 14, wherein the executable software instructs the behavioral insight server to:
assign a weighted value to behavioral insights of the subject, wherein predefined threshold parameters are based on the weighted value, and wherein the format is based at least in part on the weighted value.

17. The system of claim 16, wherein the executable software is configured to logically interface with a behavioral insight virtual assistant.

18. The system of claim 11, wherein the on-demand behavioral insights are delivered as audio.

19. The system of claim 18, wherein external hardware is accessible to the behavioral insight virtual assistant.

20. The system of claim 19, wherein on-demand behavioral insights direct behavioral insight virtual assistant external hardware interaction.
